(19) United States
(12) Patent Application Publication
(10) Pub. No.: US 2017/0267727 A1
(43) Pub. Date: Sep. 21, 2017

(54) Conjugates of pH Low Insertion Peptide and Monomethyl Auristatins in the Treatment of Solid Tumors

(71) Applicant: Lehigh University, Bethlehem, PA (US)

(72) Inventors: Damien Thevenin, Bethlehem, PA (US); Matthew K. Robinson, Rockledge, PA (US); Kelly E. Burns, Bethlehem, PA (US)

(21) Appl. No.: 15/449,318

(22) Filed: Mar. 3, 2017

Related U.S. Application Data
(60) Provisional application No. 62/303,414, filed on Mar. 4, 2016.

(51) Int. Cl. C07K 14/00; A61K 38/08; A61K 45/06; C07K 7/02

(52) U.S. Cl. CPC C07K 14/00; C07K 7/02; A61K 47/48246; A61K 38/08; A61K 45/06

(57) ABSTRACT

Constructs comprising pH low insertion peptide and variants thereof conjugated to monomethyl auristatins and analogs thereof are described. These constructs are useful, for example, in the treatment of solid tumors, including the treatment of breast cancer and prostate cancer, as well as other cancers such as pancreatic cancer, ovarian cancer, cervical cancer, uterine cancer, lung cancer, skin cancer, kidney cancer, and colon cancer. The constructs inhibit tumor cell proliferation and reduce tumor volume, particularly in a low pH tumor environment.

### CROSS-REFERENCE TO RELATED APPLICATION

This application claims the benefit of U.S. Provisional Application No. 62/303,414, filed Mar. 4, 2016, entitled: “Conjugates of pH Low Insertion Peptide and Monomethyl Auristatins in the Treatment of Solid Tumors”, the content of which is hereby incorporated herein by reference in its entirety.

### STATEMENT OF GOVERNMENT SUPPORT

The inventions herein were made, in part, with support from the National Institutes of Health, Grant Nos. CA181868 and CA06927. The United States government has certain rights in the inventions.

### REFERENCE TO SEQUENCE LISTING SUBMITTED ELECTRONICALLY

The Sequence Listing associated with the application is provided in text format in lieu of a paper copy, and is hereby incorporated by reference into the specification. The name of the text file containing the Sequence Listing is SequenceListing.txt. The text file is 7 kilobytes, was created on Jun. 5, 2017 and is being submitted electronically via EFS-Web.

### FIELD OF THE DISCLOSURE

The disclosure relates generally to the field of cancer therapy. More particularly, the disclosure relates to recombinant constructs which are based on the pH Low Insertion Peptide (pHLIP), and variants thereof, conjugated to monomethyl auristatins, and analogs thereof. Such constructs selectively target tumor tissue in vivo, and reduce tumor volume and proliferation.

### BACKGROUND

Various publications, including patents, published applications, technical articles and scholarly articles are cited throughout the specification. Each of these cited publications is incorporated by reference herein, in its entirety and for all purposes.

Cancer targeting therapies aimed at improving effectiveness and diminishing off-target cytotoxic effects have been developed and hold the promise of being curative options for defined subsets of cancers. Preclinical and clinical evidence, however, demonstrates that therapy strategies based on the targeting of specific proteins is significantly hampered by tumor heterogeneity, which can promote tumor evolution, and lead to loss of cell surface proteins and, eventually, to therapy resistance and disease progression. Moreover, targeted cancer biomarkers tend to be over-expressed in a tumor-associated, not tumor-specific manner. While over-expression provides a window of selective targeting, targeted uptake into normal tissues is seen, and has the potential to lead to unacceptable toxicity profiles. Thus, alternatives are needed to circumvent these limitations.

### SUMMARY

A construct comprising a pH Low Insertion Peptide (pHLIP) and monomethyl auristatin (MMA) comprises the amino acid sequence of SEQ ID NO: 8 or SEQ ID NO: 5 conjugated to a compound of Formula I

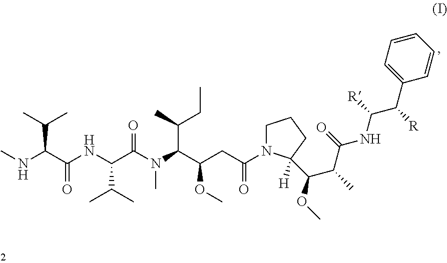

wherein R is H or OH, and R′ is C₃H₃NS, CH₃, a carboxylic acid, or an alkyl ester. The pHLIP may comprise the amino acid sequence of SEQ ID NO: 1, SEQ ID NO: 2, SEQ ID NO: 3, SEQ ID NO: 4, SEQ ID NO: 6, SEQ ID NO: 7, or SEQ ID NO: 9. The MMA conjugate may comprise MMAE, e.g., wherein the R is OH and R′ is CH₃. The MMA conjugate may comprise MMAF, e.g., wherein the R is H and R′ is acetic acid. The MMA conjugate may comprise MMAF-Ome, e.g., wherein the R is H and R′ is methyl ester. The MMA may be conjugated to pHLIP peptide via a disulfide bond. The construct may be comprised in a composition comprising a carrier, an excipient, or a carrier and an excipient. The carrier may be a pharmaceutically acceptable carrier.

These constructs and compositions thereof may be used in methods for inhibiting the proliferation of a tumor cell. Such methods generally comprise contacting a tumor cell with a construct comprising pHLIP of SEQ ID NO: 8 or SEQ ID NO: 5 conjugated to a MMA of Formula I in an amount effective to inhibit proliferation of the tumor cell. The pHLIP may comprise the amino acid sequence of SEQ ID NO: 1, SEQ ID NO: 2, SEQ ID NO: 3, SEQ ID NO: 4, SEQ ID NO: 6, SEQ ID NO: 7, or SEQ ID NO: 9. The MMA conjugate may comprise MMAE, e.g., wherein the R is OH and R′ is CH₃. The MMA conjugate may comprise MMAF, e.g., wherein the R is H and R′ is acetic acid. The MMA conjugate may comprise MMAF-Ome, e.g., wherein the R is H and R′ is methyl ester.

The tumor cell may be a breast tumor cell, a prostate tumor cell, a pancreatic tumor cell, a cervical tumor cell, a uterine tumor cell, a lung tumor cell, a skin cancer cell, a kidney cancer cell, or a colon tumor cell. The construct may be comprised in a composition comprising a carrier, such that the composition is contacted with the tumor cell.

The tumor cell preferably is in an acidic or low pH environment. The acidic or low pH environment may have a pH of less than about 7, or a pH of less than about 6.5, or a pH of less than about 6, or a pH of less than about 5.5. The pH may be from about 5 to about 6. The pH may be from about 5 to about 7. The pH may be between about 5 and about 6. The pH may be between about 5 and about 7. The method may further comprise lowering the pH in the cellular environment. The cell environment comprises the area proximal to the cell, including the extracellular milieu and, in some aspects, including stroma.

These constructs and compositions thereof may be used in methods for reducing the volume of a tumor. Such methods generally comprise contacting a tumor with a construct comprising pHLIP of SEQ ID NO: 8 or SEQ ID NO: 5 conjugated to a MMA of Formula I in an amount effective to reduce the volume of the tumor. The pHLIP may comprise the amino acid sequence of SEQ ID NO: 1, SEQ ID NO: 2, SEQ ID NO: 3, SEQ ID NO: 4, SEQ ID NO: 6, SEQ ID NO: 7, or SEQ ID NO: 9. The MMA conjugate may comprise MMAE, e.g., wherein the R is OH and R′ is CH₃. The MMA conjugate may comprise MMAF, e.g., wherein the R is H and R′ is acetic acid. The MMA conjugate may comprise MMAF-Ome, e.g., wherein the R is H and R′ is methyl ester. The tumor may comprise a breast tumor, a prostate gland tumor, a pancreatic tumor, a cervical tumor, a uterine tumor, a lung tumor, a skin cancer, a kidney cancer, or a colon tumor. The construct may be comprised in a composition comprising a carrier, such that the composition is contacted with the tumor.

The tumor preferably is in an acidic or low pH environment. The acidic or low pH environment may have a pH of less than about 7, or a pH of less than about 6.5, or a pH of less than about 6, or a pH of less than about 5.5. The pH may be from about 5 to about 6. The pH may be from about 5 to about 7. The pH may be between about 5 and about 6. The pH may be between about 5 and about 7. The method may further comprise lowering the pH in the tumor environment. The tumor environment includes the tumor itself as well as areas surrounding and proximal to the tumor.

These constructs and compositions thereof may be used in methods for treating one or more of breast cancer, prostate cancer, pancreatic cancer, cervical cancer, ovarian cancer, uterine cancer, lung cancer, skin cancer, kidney cancer, or colon cancer in a patient in need thereof, which patient has one or more of such cancers. The methods generally comprise administering to the patient a construct comprising pHLIP of SEQ ID NO: 8 or SEQ ID NO: 5 conjugated to a MMA of Formula I in an amount effective to treat the tumor. Treating the tumor may comprise inhibiting proliferation of cells of the tumor and/or reducing the volume of the tumor. The pHLIP may comprise the amino acid sequence of SEQ ID NO: 1, SEQ ID NO: 2, SEQ ID NO: 3, SEQ ID NO: 4, SEQ ID NO: 6, SEQ ID NO: 7, or SEQ ID NO: 9. The MMA conjugate may comprise MMAE, e.g., wherein the R is OH and R′ is CH₃. The MMA conjugate may comprise MMAF, e.g., wherein the R is H and R′ is acetic acid. The MMA conjugate may comprise MMAF-Ome, e.g., wherein the R is H and R′ is methyl ester. The methods may further comprise administering to the patient a chemotherapeutic agent. The methods may further comprise irradiating the tumor in the patient.

### BRIEF DESCRIPTION OF THE DRAWINGS

FIG. 1A shows sequences of the pHLIP peptides. The amino acid substitution for the variant pHLIP(D25E) is shown in bold, and the cysteine available for disulfide drug conjugation is shown underlined. FIG. 1B shows the structure of MMAE with a succinimide nitrophenyl-based linker, conjugation to pHLIP via disulfide exchange and the structure of MMAE as released intracellularly after pHLIP insertion and reduction of the disulfide bond.

FIG. 2A and FIG. 2B show pHLIP(WT)-MMAE CD spectroscopy and Trp Fluorescence, respectively. FIG. 2C and FIG. 2D show pHLIP(WT)-MMAE CD spectroscopy and Trp Fluorescence, respectively. State I corresponds to the peptides in an aqueous environment is shown in black, state II: peptides in the presence of lipid membranes at pH 7.4, shown in blue and state III: peptides in the presence of lipids at pH 5.0 is shown in red. [peptide]=7 μM and peptide-lipid ratio=1:300 with POPC liposomes.

FIG. 3A, FIG. 3C and FIG. 3E show HeLa cells treated with pHLIP(WT)-MMAE, pHLIP(D25E)-MMAE, and MMAE(linker), respectively. FIG. 3B, FIG. 3D and FIG. 3F show MDA-MB-231 cells treated with pHLIP(WT)-MMAE, pHLIP(D25E)-MMAE, and MMAE(linker), respectively. Black columns represent cells treated at pH 7.4 and cells treated at low pH are shown in grey. For each condition, 3,000 cells/well (96-well plate) were seeded, allowed to adhere overnight, treated for 2 hours, washed once with media and grown for 72 hour in complete medium at physiologic pH. Cell viability was assessed with the MTT assay. All measurements were normalized to the media control (0 μM, pH 7.4), as 100% cell viability. Results are shown as mean±SD (n=6-9). To assess statistical significance two-tailed Student's t test analyses at 95% confidence level were performed for the comparison of pH 7.4 and pH 5.0 treatments (**** p<0.0001; *** p=0.0004; ** p=0.0009 and * p=0.011). For FIG. 3E and FIG. 3F, none of the differences between treatment at pH 7.4 and pH 5.0 were statistically different (p>0.01).

FIG. 4A and FIG. 4B show pHLIP-MMAE conjugates do not disrupt the cell membrane. The integrity of the plasma membrane is assessed by the uptake of trypan blue dye. 3,000 cells/well of HeLa (FIG. 4A) and MDA-MB-231 (FIG. 4B) cells were seeded, allowed to adhere overnight and treated with 10 μM conjugates or media alone (no peptide) for 2 hours at pH 7.4 (black bars) or pH 5.0 (grey bars). Cells were detached and counted based on trypan blue uptake with a hemacytometer: % of intact cells corresponds to the number of cells not showing any dye uptake over the total number of cells. Results are shown as mean±SD (n=4).

FIG. 5A, FIG. 5B, and FIG. 5C show optical imaging of Alexa705-pHLIP(WT)-MMAE in vivo targeting. Clearance and tumor targeting of Alexa750-pHLIP(WT)-MMAE in NCr nu/nu mice harboring MDA-MB-231 tumor xenografts visualized over a 48 hour time course post-injection. In FIG. 5A imaging was performed with the minimum and maximum average radiant efficiency set to 1.1e7 and 1.4e8 [p/s/cm²/sr]/[μW/cm²], respectively for all time points. Color intensity reflects absolute levels of probe. Arrow and chevron noted on 28 hr time point in FIG. 5A denote position of tumor and kidney, respectively. The third region displaying relatively high fluorescence corresponds to the epi-fluorescence originating from the region corresponding to the knee joint. In FIG. 5B contrast is set in automatic mode to optimize visualization of tumor targeting at each time point FIG. 5C shows quantitation of average radiant efficiency [p/s/cm²/sr]/[μW/cm²] for ROIs drawn around tumor, kidney, and background (blood pool). Results are shown as mean±SD.

FIG. 6A and FIG. 6B show inhibition of HeLa (FIG. 6A) and MDA-MB-231 (FIG. 6B) cell proliferation by free MMAE modified with a succinimide nitrophenyl-based linker (MMAE+linker) and by unmodified MMAE. For each cell assay 3,000 cells/well were seeded, allowed to adhere overnight and incubated with either compound for 72 hours in complete medium at physiologic pH. Cell viability was determined with the MTT assay. All measurements were normalized to the 1% DMSO controls. Results are shown as mean±SD (n=6). Data were fitted with a sigmoidal dose response function in Prism for Mac (GraphPad Software, Inc.).

FIG. 7A and FIG. 7B show the pHLIP peptide variants are not cytotoxic. (FIG. 7A) HeLa cells and (FIG. 7B) MDAMB-231 treated with 10 μM of either pHLIP(WT) or pHLIP(D25E) at pH 7.4 (black bars) or pH 5.0 (grey bars). Cell viability was assessed with the MTT assay, and all measurements were normalized to the media control (0 pH 7.4), as 100% cell viability. Results are shown as mean±SD (n=6-9).

FIG. 8A, FIG. 8B, and FIG. 8C show Purity check and Chemical stability of pHLIP-MMAE conjugates. (FIG. 8A) and (FIG. 8B): HPLC traces of pHLIP(WT)-MMAE and pHLIP(D25E)-MMAE, respectively: Purified conjugates (upper panels), 10 μM of conjugate in cell media (middle panels) and after incubation in cell media for 2 h at pH 5.0 and 37° C. (lower panels). The small peak observed around 27 min in (FIG. 8B) upper panel corresponds to some residual free pHLIP (<15%), which is known to not be cytotoxic (FIG. 7). (FIG. 8C) HPLC traces of Alexa750-pHLIP(WT)-MMAE: Purified conjugate (upper panel), 10 μM Alexa 750-pHLIP(WT)-MMAE before (upper panel) and after 48 h incubation in mouse serum at 37° C. (lower panel). Purity checks and chemical stability runs were performed on different column and with different flow rate, explaining the difference in retention times and peak broadness (see Experimental Section). Because of the presence of many components in cell medium and mouse serum, only portion of the HPLC trace is shown in the middle and lower panel traces for clarity. No significant degradation was observed for any of the pHLIP conjugates: For each HPLC run, every peak was collected and checked by mass spectroscopy. In all cases, the pHLIP-MMAE conjugate was found intact, while neither pHLIP nor MMAE alone was found in any other peaks in the entire retention time range by MALDITOF.

FIG. 9A-FIG. 9D inhibition of cell growth with pHLIP-MMAF variants. HeLa cells treated with pHLIP(WT)-MMAF (FIG. 9A), pHLIP(D25E)-MMAF (FIG. 9B), pHLIP(P20G)-MMAF (FIG. 9C), and pHLIP(R11Q)-MMAF (FIG. 9D). Circles represent cells treated at pH 7.4 and squares represent cells treated at pH 5.0. Results are shown as the mean±SEM (n=6-9).

FIG. 10A-FIG. 10C show the therapeutic efficacy in mice. HeLa (ATCC#CCL-2) were injected subcutaneously into the inguinal space of female Ncr nu/nu mice. Tumors were allowed to develop until they were approximately 75 mm³ in size. On day 0, animals were randomized into cohorts with statistically similar tumor sizes; pHLIP-MMAF (76.8+/−19.3 mm³; n=8) and vehicle control (73+/−15.6 mm³; n=9) cohorts. On days 1, 3, 5, and 8, animals were injected intravenously with 200 microliters of either pHLIP-MMAF (20 microM in PBS+1% DMSO) or vehicle control (PBS+1% DMSO). Tumors were measured with calipers on routine basis for approximately 3 weeks and tumor volumes calculated with the formula: length×width×(height×0.5). Animals were euthanized when tumors reached a volume of 500 mm³. FIG. 10A depicts tumor growth as a function of initial tumor volume. FIG. 10B depicts tumor volume. FIG. 10C depicts overall survival.

FIG. 11 shows HeLa pHLIP(WT)-MMAF compiled data minus outliers; error bars=SE; n=6-9.

FIG. 12A shows the effects of treatment of A431 cells with pHLIP(WT)-MMAF conjugate after two hours of treatment, followed by a wash, and a 72 hour recovery period. FIG. 12B shows the effects of treatment of A431 cells with free MMAF after two hours of treatment, followed by a wash, and a 72 hour recovery period.

FIG. 13 shows the effects of treatment with 1 mg/kg of pHLIP-MMAF (ip) on A431 xenograft tumor size.

FIG. 14A and FIG. 14B compile an immunohistochemistry analysis of A431 tumors stained for Ki67 as a secondary marker of drug efficacy with results of the non-treated cohort shown in FIG. 14A and the treated cohort in FIG. 14B. Treatment with pHLIP-MMAF was capable of slowing tumor cell proliferation as judged by the level of Ki-67 staining. These results were significant (P≦0.05) as determined by Wilcoxon rank sum test.

### DETAILED DESCRIPTION

Various terms relating to aspects of the disclosure are used throughout the specification and claims. Such terms are to be given their ordinary meaning in the art, unless otherwise indicated. Other specifically defined terms are to be construed in a manner consistent with the definition provided herein.

The terms “subject” or “patient” are used interchangeably and refer to any animal. Mammals are preferred, and include companion and farm mammals, as well as rodents, including mice, rabbits, and rats, and other rodents. Primates are more preferred, and human beings are highly preferred.

As used herein, the singular forms “a,” “an,” and “the” include plural referents unless expressly stated otherwise.

It has been observed in accordance with the disclosure that monomethyl aruistatins (MMA) may be selectively targeted to tumors in vivo by conjugating the molecule to the pH low insertion peptide (pHLIP), with an attendant inhibition of proliferation and with a reduction of tumor volume. Tumor targeting was enhanced by low pH in the tumor environment. It was further observed that the conjugation of the monomethyl auristatins to the pHLIP peptide reduced the toxicity of the monomethyl auristatin molecule. In vivo experiments correlated well with in vitro tests. Accordingly, the disclosure features constructs comprising pHLIP and variants thereof conjugated to monomethyl auristatins. The monomethyl auristatins may comprise structural analogs to Dolastatin 10. The disclosure also features methods for using such constructs in the treatment of cancer.

Dolastatin 10 has the following chemical Formula I:

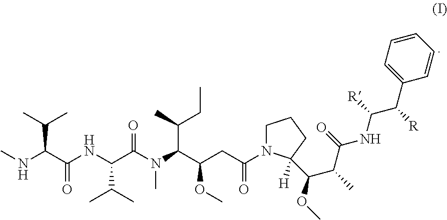

Monomethyl auristatin (MMA) derivatives of Dolastatin 10 have the following characteristics:

| Compound | R | R′ | Log P o/w | IC₅₀ (nm) |
|---|---|---|---|---|
| Dolastatin 10 | H | C₃H₃NS | 3.4 | 0.1 |
| MMAE | OH | CH₃ | 2.2 | 0.1-2 |
| MMAF | H | COOH | 0.7 | 105-250 |
| MMAF-Ome | H | COOMe | 2.8 | .001 |

Thus, in Formula I, R may comprise H or OH. In Formula I, R′ may comprise C₃H₃NS, or may comprise an alkyl such as methyl (MMAE), or may comprise a carboxylic acid such as acetic acid (MMAF), or may comprise an alkyl ester such as methyl ester (MMAF-OMe).

The pHLIP peptide of the construct may comprise the consensus amino acid sequence of SEQ ID NO: 8. The peptide may comprise the wild type (WT) amino acid sequence of SEQ ID NO: 1. The pHLIP peptide may comprise amino acid substitutions or replacements, relative to the WT, which retain or improve the biological properties of the WT pHLIP, including the capacity to insert into the surface of cells. The amino acid variations are preferably conservative, but may be non-conservative. In some aspects, the pHLIP peptide may comprise variants having at least about 90%, at least about 92%, at least about 95%, at least about 96%, at least about 97%, at least about 98%, or at least about 99% sequence identity with the amino acid sequence of SEQ ID NO: 1 or SEQ ID NO: 7.

In some aspects, the pHLIP amino acid sequence may further comprise a cysteine residue at the c-terminus, for example, SEQ ID NO: 7 (wild type+c-terminal cysteine) or SEQ ID NO: 9 (consensus+c-terminal cysteine). In some aspects, the pHLIP has the amino acid sequence of SEQ ID NO: 2 (D25E), SEQ ID NO: 3 (P20G), SEQ ID NO: 4 (R11Q), or SEQ ID NO: 5 (D14Gla/D25Aad). In some aspects, the pHLIP comprises an insertion of an Ala between D14 and W15 of the WT molecule and has SEQ ID NO: 5.

The pHLIP peptide may comprise post-translational modifications or moieties. For example, the peptide may be methylated, acetylated, glycosylated, sulfated, phosphorylated, carboxylated, or amidated, or comprise and other moieties that are known in the art. Moieties include any chemical group or combinations of groups commonly found on the pHLIP peptide in nature, or otherwise added to proteins by particular recombinant expression systems, including prokaryotic and eukaryotic expression systems.

The construct comprises a MMA molecule conjugated to the pHLIP peptide. The MMA molecule may comprise Formula I or analog thereof. Conjugation may be according to any suitable chemical conjugation, and is preferably covalent. In some aspects, the conjugation is by way of a disulfide bond formed between a c-terminal cysteine on pHLIP and a sulfur moiety on the MMA molecule. In some aspects, the conjugation is by way of a linker such as a citrulline-valine (Cit-Val) linker or a phenylalanine-lysine (Phe-Lys) linker, and such a linker may further include a spacer such as a p-aminobenzylcarbamate or malemidocaproyl spacer. In some aspects, the spacer is used without a linker. The MMA molecule may comprise MMAE, MMAF, or MMAF-OMe. MMAF is highly preferred.

The construct may optionally be labeled. Labeled constructs may find use in diagnostic or basic research applications, in addition to therapeutic modalities as described or exemplified herein. Such labels/conjugates can be detectable, such as fluorochromes, radiolabels, enzymes, fluorescent proteins, and biotin.

The construct may be formulated in a composition comprising at least one of any suitable auxiliary, such as, but not limited to one or more, diluents, binders, stabilizers, buffers, salts, lipophilic solvents, preservatives, adjuvants, or other suitable carrier and/or excipient. Pharmaceutically acceptable auxiliaries are preferred. The compositions may comprise a carrier such as a pharmaceutically acceptable carrier. Pharmaceutically acceptable carriers include any media that does not interfere with the biological activity of the construct and preferably is not toxic to a patient to which it is administered. The carrier may be an aqueous solution, such as water, saline, or alcohol, or a physiologically compatible buffer, such as Hanks's solution, Ringer's solution, or physiological saline buffer. The carrier may contain formulatory agents, such as suspending, stabilizing and/or dispersing agents.

The compositions may be formulated for administration to a patient in any suitable dosage form. The compositions may be formulated for oral, buccal, nasal, transdermal, parenteral, injectable, intravenous, subcutaneous, intramuscular, rectal, or vaginal administrations. The compositions may be formulated in a suitable controlled-release vehicle, with an adjuvant, or as a depot formulation.

Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions.

The pHLIP-MMA constructs are useful, for example, as cancer therapeutics because the constructs selectively target to tumor cells, particularly where the tumor comprises an acidic environment. The construct may inhibit proliferation of tumor cells, reduce tumor volume, or inhibit proliferation of tumor cells and reduce tumor volume. Non-limiting examples of tumors that can be treated using these constructs include breast tumors, prostate tumors, pancreatic tumors, cervical tumors, uterine tumors, lung tumors, skin tumors, kidney tumors, and colon tumors. Breast tumors and prostate tumors are preferred targets.

The pHLIP-MMA constructs, when contacted with tumor cells, inhibit proliferation of the tumor cells. Thus, methods for inhibiting proliferation of tumor cells are provided. The methods may be carried out in vitro, in vivo, ex vivo, or in situ.

Methods for inhibiting proliferation of a cancer cell comprise contacting a cancer cell with a pHLIP-MMA conjugate (construct) in an amount effective to inhibit proliferation of the cancerous cell. Methods for reducing tumor volume comprise contacting a tumor with a pHLIP-MMA conjugate (construct) in an amount effective to reduce the tumor volume. In any such methods, the construct may comprise any pHLIP-MMA conjugate described or exemplified herein. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The conjugate may be in a composition comprising a carrier such as a pharmaceutically acceptable carrier. The methods may be used to inhibit the proliferation of any cancer cell to which the pHLIP peptide may be inserted. The cancer cell may comprise a breast cancer cell, a prostate cancer cell, a pancreatic cancer cell, a cervical cancer cell, an ovarian cancer cell, a uterine cancer cell, a lung cancer cell, a skin cancer cell, a kidney cancer cell, or a colon cancer cell. Breast cancer cells and prostate cancer cells are preferred. The methods may be used to inhibit the proliferation of any cancer cell from any solid tumor. The cells may be in an acidic environment, such as a pH of from about 5 to about 7, or from about 5 to about 6. The methods may further comprise lowering the pH of the cellular environment.

The cells may be comprised in a tumor. For example, the construct may be contacted with cells in a tumor, thereby inhibiting proliferation of cells within the tumor, thereby treating the tumor. The tumor may comprise a tumor of the breast, a tumor of the prostate, a tumor of the pancreas, a tumor of the ovary, a tumor of the uterus, a tumor of the cervix, a tumor of the lung, a tumor of the skin, a tumor of the kidney, or a tumor of the colon. In some aspects, the methods may further comprise administering to the tumor a chemotherapeutic agent. In some aspects, the methods may further comprise irradiating the tumor.

In some aspects, the methods comprise treating breast cancer. The methods comprise administering to a breast cancer patient in need thereof a construct comprising a pHLIP peptide conjugated to a MMA, in an amount effective to treat a tumor of the breast of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the breast tumor in the patient.

In some aspects, the methods comprise treating prostate cancer. The methods comprise administering to a prostate cancer patient in need thereof a construct comprising a pHLIP peptide conjugated to a MMA, in an amount effective to treat a tumor of the prostate gland of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the prostate tumor in the patient.

In some aspects, the methods comprise treating ovarian cancer. The methods comprise administering to an ovarian cancer patient in need thereof a construct comprising a pHLIP peptide conjugated to a MMA, in an amount effective to treat a tumor of the ovary of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the ovary tumor in the patient.

In some aspects, the methods comprise treating cervical cancer. The methods comprise administering to a cervical cancer patient in need thereof a construct comprising a pHLIP peptide conjugated to a MMA, in an amount effective to treat a tumor of the cervix of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the cervical tumor in the patient.

In some aspects, the methods comprise treating uterine cancer. The methods comprise administering to a uterine cancer patient in need thereof a construct comprising a pHLIP peptide conjugated to a MMA, in an amount effective to treat a tumor of the uterus of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the uterine tumor in the patient.

In some aspects, the methods comprise treating lung cancer. The methods comprise administering to a lung cancer patient in need thereof a construct comprising a pHLIP peptide conjugated to a MMA, in an amount effective to treat a tumor of the lung of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the lung tumor in the patient.

In some aspects, the methods comprise treating skin cancer. The methods comprise administering to a skin cancer patient in need thereof a construct comprising a pHLIP peptide conjugated to a MMA, in an amount effective to treat a tumor of the skin of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the skin tumor in the patient.

In some aspects, the methods comprise treating kidney cancer. The methods comprise administering to a kidney cancer patient in need thereof a construct comprising a pHLIP peptide conjugated to a MMA, in an amount effective to treat a tumor of the kidney of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the kidney tumor in the patient.

In some aspects, the methods comprise treating colon cancer. The methods comprise administering to a colon cancer patient in need thereof a construct comprising a pHLIP peptide conjugated to a MMA, in an amount effective to treat a tumor of the colon of the patient. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. The construct may be in a composition comprising a carrier such as a pharmaceutically acceptable carrier. The methods may further comprise administering a chemotherapeutic agent to the patient and/or irradiating the colon tumor in the patient.

In any of the methods described or exemplified herein, the construct (pHLIP-MMA conjugate) may be administered directly to the tumor, including any substructure or location in the tumor. The construct may be administered proximally to the tumor, including any location not directly in, but proximal to the tumor such that the construct diffuses to and/or into the tumor, or can be actively targeted to the tumor. The construct may be administered distally to the tumor, such that the construct diffuses to and/or into the tumor. Diffusion may be passive (e.g., via blood flow). Proximally or distally administered constructs or compositions comprising the construct may also be actively targeted to the tumor.

Use of a pHLIP-MMAe conjugate (construct) in the treatment of cancers/tumors are provided. The construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred. Any of the pHLIP-MMA conjugates/constructs described or exemplified herein may be for use as a medicament. Any of the pHLIP-MMA conjugates/constructs described or exemplified herein may be for use in the manufacture of a medicament.

Any of the pHLIP-MMA conjugates/constructs described or exemplified herein may be for use in treating breast cancer. Any of the pHLIP-MMA conjugates/constructs described or exemplified herein may be for use in treating prostate cancer. Any of the pHLIP-MMA conjugates/constructs described or exemplified herein may be for use in treating pancreatic cancer. Any of the pHLIP-MMA conjugates/constructs described or exemplified herein may be for use in treating ovarian cancer. Any of the pHLIP-MMA conjugates/constructs described or exemplified herein may be for use in treating cervical cancer. Any of the pHLIP-MMA conjugates/constructs described or exemplified herein may be for use in treating uterine cancer. Any of the pHLIP-MMA conjugates/constructs described or exemplified herein may be for use in treating lung cancer. Any of the pHLIP-MMA conjugates/constructs described or exemplified herein may be for use in treating skin cancer. Any of the pHLIP-MMA conjugates/constructs described or exemplified herein may be for use in treating kidney cancer. Any of the pHLIP-MMA conjugates/constructs described or exemplified herein may be for use in treating colon cancer.

Kits for use in practicing the methods described and exemplified herein are provided. The kits may be used to supply the pHLIP-MMA conjugates/constructs, or a composition thereof, as well as methods for treating a tumor with the construct or composition thereof. The kit may comprise a device for injecting the construct or composition thereof into a patient, including but not limited to a syringe and needle, or catheter. In the kit, the construct may comprise a pHLIP having the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The MMA may comprise Formula I or any analog or homolog thereof. MMAE, MMAF, and MMAF-Ome are preferred.

### EXAMPLES

The following examples are provided to describe the invention in greater detail. They are intended to illustrate, not to limit, the invention.

### Example 1

### Materials and Methods for Targeting Breast Tumors with pHLIP-MMAE Conjugate

Materials. N-Hydroxybenzotriazole (HOBt), o-benzotriazol-N,N,N,N′,N′-tetramethyluronium hexafluorophosphate (HBTU), and all N-fluorenyl-9-methoxycarbonyl (Fmoc) protected L-amino acids were purchased from GL Biochem Ltd. H-Rink Amide-ChemMatrix solid support resin was from PCAS BioMatrix Inc. Diisopropylethylamine (DIEA), piperazine, N, N-dimethylformamide (DMF), dichloromethane (DCM), trifluoroacetic acid (TFA), methanol, acetonitrile, dimethyl sulfoxide (DMSO) and Dulbecco's modified Eagle's medium (DMEM) were all from Thermo Fisher Scientific Inc. Fetal Bovine Serum (FBS) was purchased from Atlanta Biologicals Inc. Penicillin-Streptomycin was purchased from Sigma-Aldrich. Monomethylauristatin E (MMAE), and pyridyldithiol-activated MMAE (Py-ds-Prp-MMAE) were from Concortis. 1-palmitoyl-2-oleoyl-sn-glycero-3-phosphocholine (POPC) was from Avanti Polar Lipids Inc. 3-(4,5-dimethylthiazol-2-yl)-2,5-diphenyltetrazolium bromide (MTT) was purchased from EMD Millipore. AlexaFluor 750 NHS ester was purchased from Life Technologies.

Solid-Phase Peptide Synthesis. Two variants of pHLIP were used in this study: pHLIP-Cys (pHLIP(WT)) with a cysteine residue at its C terminus (SEQ ID NO: 7) and pHLIP-D25E-Cys (pHLIP(D25E)) (SEQ ID NO: 2) where the transmembrane Asp25 residue is replaced with Glu. pHLIP-Cys was either purchased from GL Biochem Ltd (with a free C-terminal carboxylic acid) or prepared by solid-phase synthesis. pHLIP(D25E) was prepared in the laboratory. Briefly, peptides were prepared by Fmoc solid-phase synthesis, using H-Rink Amide resin affording an amidated C-terminus and purified via reverse-phase high performance liquid chromatography (RP-HPLC) (Phenomenex Luna prep 10μ250×21.20 mm C8; flow rate 10 mL/min; phase A: water 0.1% TFA; phase B: acetonitrile 0.1% TFA; gradient 60 min from 95/5 A/B to 0/100 A/B). The purity of the peptides was determined by RP-HPLC (Agilent Zorbax Eclipse 5 μm 4.6×50 mm XDB-C8; flow rate 1 mL/min; phase A: water 0.01% TFA; phase B: acetonitrile 0.01% TFA; gradient 45 min from 95/5 A/B to 0/100 A/B) and their identity was confirmed via matrix-assisted laser desorption ionization time of flight (MALDI-TOF) mass spectrometry. No significant difference in behavior (e.g., secondary structure, pH-dependent properties) were observed between C-term amidated and free carboxylated peptides (data not shown).

Preparation of pHLIP-MMAE Constructs. Each pHLIP peptide was dissolved in DMSO to a concentration of 1 mM, followed by the addition of 1.5 eq. of Py-ds-Prp-MMAE in DMSO. To facilitate disulfide exchange, 100 μL of 1 M Tris buffer, pH 8.0 was added to the solution and allowed to mix at room temperature for 2-3 hours. The N-terminally modified AlexaFluor 750-pHLIP(WT)-MMAE conjugate was obtained by dissolving 1 mg of the NHS activated AlexaFluor 750 in 200 μL of DMF, followed by the addition of 1 eq. of pHLIP(WT)-MMAE in DMF, in presence of DIEA affording amide bond conjugation to the N-terminal amine of pHLIP. The desired pHLIP conjugates were isolated using the same techniques described for the pHLIP peptides. The purity of the peptide-drug conjugates was determined by RP-HPLC as listed, and their identity was confirmed by MALDI-TOF MS: pHLIP(WT)-MMAE: purity >98%; calculated (M+H+)=5046; found (M+H+)=5047. pHLIP(D25E)-MMAE: purity >85%; calculated (M+H+) 5031; found (M+H+)=5032. Alexa750-pHLIP(WT)-MMAE: purity >95%; calculated (M+H+) 5915; found (M+H+)=5912. The conjugates were quantified at 280 nm by UV/Vis absorbance spectroscopy using the molar extinction coefficient of pHLIP (13,940 M-1 cm-1) and lyophilized to 10-8 mole aliquots.

Preparation of POPC Liposomes. Ten milligrams of POPC in chloroform were dried and then held under vacuum overnight. The dried lipid film was rehydrated with 1 ml of 5 mM sodium phosphate, pH 8.0, and mixed by vigorous vortex. The resulting multilamellar vesicle solution was freeze-thawed in liquid nitrogen for seven cycles and were extruded through a polycarbonate membrane with 100 mm diameter pores using a mini extruder (Avanti Polar Lipids) to produce large unilamellar vesicles. Liposomes were used immediately following their preparation. Lipid concentration was checked using the Marshall's assay and the size distribution was verified by dynamic light scattering analysis performed at a scattering angle of 90° using an ALV/GSC-3 goniometer system equipped with ALV/ALV-7004 correlator (ALV-GmbH, Langen, Germany).

Sample preparation for Circular Dichroism and Tryptophan Fluorescence Measurements. Prior to CD or fluorescence measurements, lyophilized aliquots of pHLIP constructs were resuspended to 20 μM with 5 mM sodium phosphate, pH 8.0, and incubated for 1 hour at room temperature. This stock solution was diluted to a final concentration of 7 μM and, when appropriate, incubated with POPC liposomes at a 1:300 peptide-to-lipid molar ratio for 30 minutes at room temperature. Subsequently, samples were adjusted to the desired experimental pH values with the addition of aliquots of an HCl solution to elicit pH-dependent insertion. Samples were allowed to equilibrate at the desired pH for 30 minutes at room temperature before spectroscopic measurements.

Tryptophan Fluorescence Spectroscopy. All measurements were carried out using a Cary Eclipse Fluorescence Spectrophotometer (Varian), and performed at 25° C. with pHLIP construct concentrations equal to 7 μM. Samples were excited at 295 nm and the emission spectra were taken from 300 to 500 nm, with the slit widths for emission and excitation both set 5 nm.

Circular Dichroism Spectroscopy. Far-UV CD spectra of pHLIP constructs were recorded on Jasco J-815 CD spectrometer equipped with a Peltier thermal-controlled cuvette holder (Jasco, Inc.). All measurements were performed in 0.1 mm quartz cuvette at 25° C. with pHLIP constructs concentrations equal to 7 μM. CD intensities are expressed in mean residue molar ellipticity [θ] calculated from the following equation:

[θ]=θobs/(10×1 cn)

where, θobs is the observed ellipticity in millidegrees (cm² dmol⁻¹), l is the optical path length in centimeters, c is the final molar concentration of the peptides, and n is the number of amino acid residues. Samples were measured in a 0.1 cm path length quartz cuvette and raw data were acquired from 260 nm to 190 nm at 1 nm intervals with a 100 nm/min scan rate, and at least five scans were averaged for each sample. The spectrum of POPC liposomes was subtracted out from all construct samples.

Cell Culture. Human cervical adenocarcinoma HeLa cells and human breast adenocarcinoma MDA-MB-231 cells were cultured in Dulbecco's modified Eagle's medium (DMEM supplemented with 10% FBS, 100 U/mL penicillin, and 0.1 mg/mL streptomycin in a humidified atmosphere of 5% CO₂ at 37° C.

Anti-proliferation Assay. HeLa and MDA-MB-231 cells were seeded in 96-well plates at a density of 3,000 cells/well and incubated overnight. Before treatment, construct aliquots were solubilized in an appropriate volume of DMEM without FBS (pH 7.4)—so that upon pH adjustment the desired treatment concentration is obtained—and gently sonicated for 30-60 seconds using a bath sonicator (Branson Ultrasonics). After removal of cell media, this treatment solution was added to each well and incubated at 37° C. for 5-10 minutes. The pH was then adjusted to the desired value using a pre-established volume of DMEM, pH 2.0 buffered with citric acid (final volume=50 μL) and the plate was incubated at 37° C. for 2 hours. After treatment, the media was removed, cells were washed once with 100 μL of complete DMEM, and 100 μL of complete medium was added to each well before returning the plate to the incubator. Treatment solutions were collected and their pH values measured using a micro-combination pH probe (Microelectrodes, Inc.). For physiologic pH treatments a small down-drift (˜0.2 pH unit) was usually observed, whereas an up-drift was observed for low pH treatments (e.g., pH 7.4→pH 7.2, and pH 5.0→pH 5.2). Cell viability was determined after 72 hours using the colorimetric MTT assay. Briefly, 10 μL of a 5 mg/mL MTT stock solution was added to the treated cells and incubated for 2 hours at 37° C. The resulting formazan crystals were solubilized in 200 μL DMSO and the absorbance measured at 580 nm using an Infinite 200 PRO microplate reader (Tecan). Cell viability was calculated against control cells treated with media at physiologic pH.

Cell Membrane Integrity Assay. HeLa and MDA-MB-231 cells were seeded in 96-well plates at a density of 3,000 cells/well and incubated until confluent (˜72 hours). Before treatment, construct aliquots were solubilized in an appropriate volume of DMEM without FBS (pH 7.4) so that upon pH adjustment 10 μM is obtained. Cell media was removed, the treatment solution was added to each well and cell were incubated at 37° C. for 5-10 minutes. The pH was then adjusted to the desired value using a pre-established volume of DMEM, pH 2.0 buffered with citric acid (final volume=50 μL). The plate was incubated at 37° C. for 2 hours. After treatment, the media was removed, cells were detached using trypsin and counted based on trypan blue uptake using a hemacytometer.

Construct Stability HPLC in Cell Media and Mouse Serum. pHLIP(WT)-MMAE and pHLIP(D25E)-MMAE were solubilized in DMEM without FBS (pH 7.4) to obtain a 10 μM solution, and gently sonicated for 30-60 seconds using a bath sonicator. Peptide solutions were incubated at either pH 7.4 or pH 5.0 (37° C. and 5% CO₂) and 100 μL aliquots were monitored via RP-HPLC at various time points (Agilent Eclipse XDB-C18 5 μM 9.4×250 mm; flow rate 2 mL/min; phase A: water 0.01% TFA; phase B: acetonitrile 0.01% TFA; gradient 45 min from 95/5 A/B to 0/100 A/B). HPLC peaks were collected and identified via MALDI-TOF MS. The stability of Alexa750-pHLIP(WT)-MMAE was determined in mouse serum by solubilizing the construct to incubating it at 37° C. and monitoring via RP-HPLC at specific time points.

Tumor Targeting In Vivo. Tumor targeting of Alexa750-pHLIP(WT)-MMAE was evaluated in NCr nu/nu mice harboring MDA-MB-231 tumor xenografts (n=3). Tumor cells were implanted subcutaneously (3×10⁶/animal) and allowed to grow until tumors reached volumes of approximately 100 mm³. Alexa750-pHLIP(WT)-MMAE was then administered intravenously (100 μL, 5 μM in PBS pH 7.2) and its uptake in tumor and normal tissues was monitored over time by optical imaging on an IVIS Spectrum (Perkin Elmer). Average Radiant Efficiency (ARE, expressed as [p/s/cm²/sr]/[μW/cm²]) was calculated for regions of interest (ROIs) drawn around regions encompassing tumor, kidney, and background blood pool.

### Example 2

### Results of Targeting Breast Tumors with pHLIP-MMAE Conjugate

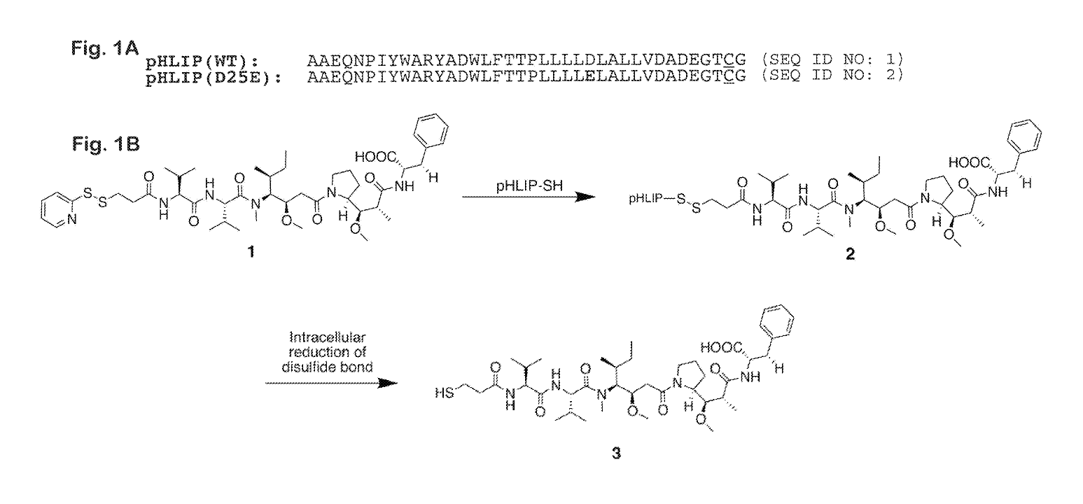

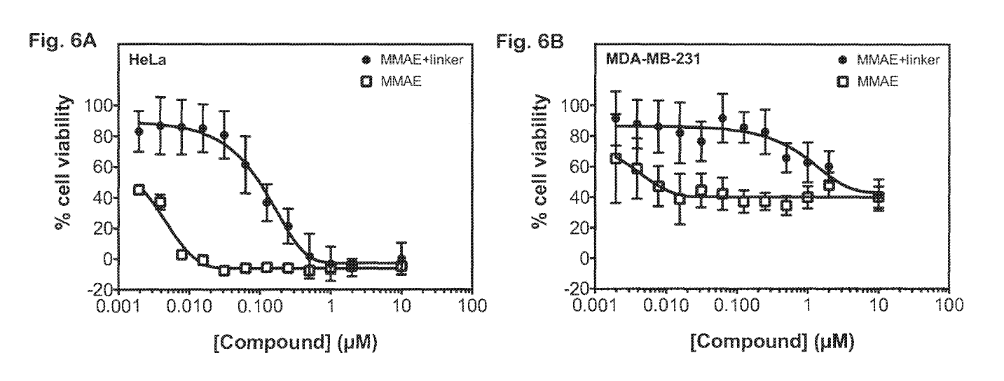

Synthesis of pHLIP-drug Conjugates. Auristatins are synthetic analogs of the natural peptide dolastatin 10, a highly potent microtubule depolymerization agent originally isolated from the sea organism Dolabella auricularia. A number of synthetic derivatives have been produced, such as monomethyl auristatin E (MMAE), monomethyl auristatin F (MMAF), and monomethyl auristatin F-OMe (MMAF-OMe), with the goal to optimize properties such as solubility, potency, pharmacokinetics, and chemical linkers for delivery vehicles. Auristatins have an IC₅₀ that is 52- and 197-fold more toxic than the clinically relevant chemotherapies vinblastine and doxorubicin, respectively. However, a targeting mechanism is necessary to deliver these potent cytotoxic agents to reduce their off-target effects and to increase their concentrations into tumors. For instance, ADCs have exploited the targeting specificity of monoclonal antibodies to selectively deliver MMAE to tumors in an effort to increase the therapeutic window of these highly toxic agents. Toward this end, MMAE, modified with a succinimide nitrophenyl-based linker, was conjugated to the C-terminus of pHLIP and pHLIP(D25E) (FIG. 1A) via a cleavable disulfide bond and purified the conjugates by HPLC and determined via MALDI-TOF MS. This linking strategy allows for the release of MMAE into the cytoplasm of cancer cells after translocation and reduction of the disulfide bond (FIG. 1B). Cleavage of the disulfide bond results in release of an N-terminally modified form of MMAE (FIG. 1B). The resulting MMAE derivative and the parental MMAE exhibit IC₅₀˜125 nM and 4 nM against HeLa cells, respectively, when incubated with the cells for 72 hours (FIG. 6A).

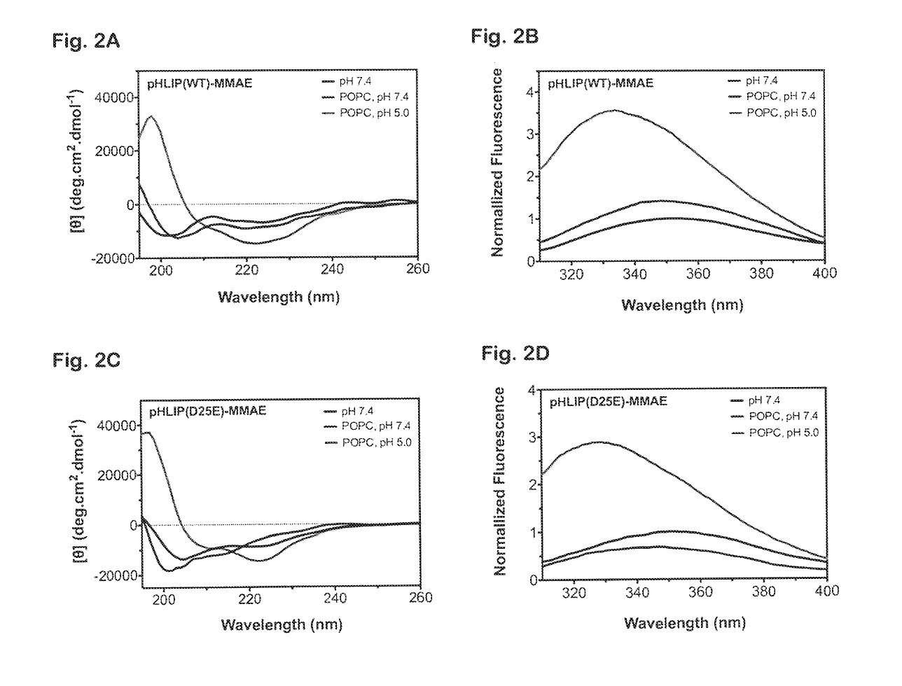

Biophysical Characterization of the Interaction of pHLIP-MMAE Conjugates with Lipid Bilayers. Prior to evaluating the pHLIP-mediated translocation of MMAE into cancer cells, the interaction of the pHLIP-MMAE conjugates with lipid bilayers was studied (FIG. 2A-FIG. 2D). Far-UV circular dichroism spectroscopy (CD) was used to determine the secondary structure of pHLIP-MMAE conjugates in the presence of POPC liposomes at both normal and low pH. FIG. 2A shows the typical pH-dependent transition of pHLIP from an unstructured configuration at pH 7.4 (middle line) to an α-helical structure when the pH is lowered to 5.0 (top line). Tryptophan fluorescence was employed to determine the insertion of pHLIP into the vesicle bilayers based on the sensitivity of the fluorescence emission of two tryptophan residues present in the sequence of pHLIP to the polarity of the environment. At pH 7.4, the Trp fluorescence emission maxima of pHLIP is centered at 348 nm (FIG. 2B, middle line), reflecting the exposure of Trp residues to polar, aqueous environments. Lowering the pH to 5.0 results in a 14 nm max blue shift (from 348 nm to 334 nm; top line), which is characteristic of Trp residues buried in hydrophobic environments, suggesting pHLIP's insertion into the membrane. Taken together, these results indicate that the presence of MMAE at the C-term of pHLIP does not significantly affect the pH-mediated insertion of pHLIP.

With the goal of optimizing the targeting and delivery of MMAE by pHLIP, the variant pHLIP(D25E) (FIG. 1A), which has a higher pKa of insertion than native pHLIP(WT) (6.49 vs. 6.0), was considered. The higher pKa of insertion may match better the extracellular pH of tumors (pH 6.5 to 6.9) and favor pHLIP insertion and MMAE translocation into cells. Similarly to pHLIP(WT)-MMAE, CD and tryptophan fluorescence measurements indicate that conjugating MMAE to pHLIP(D25E) does not interfere with pHLIP's characteristic shape-shifting behavior. Indeed, a transition from an unstructured conformation to an α-helix, and a clear blue shift (from 347 nm to 329 nm) are observed by CD (FIG. 2C) and Trp fluorescence (FIG. 2D), respectively. Thus, both pHLIP(WT)-MMAE and pHLIP(D25E)-MMAE conjugates can insert in lipid bilayers under acidic conditions to form stable transmembrane α-helices in a manner similar to that of pHLIP alone. It is believed that the pKa of insertion of the pHLIP-MMAE conjugates does not differ significantly from pHLIP alone (pKa˜6) because 1) MMAE has no ionizable group that could influence the pKa of insertion, and 2) conjugation of phalloidoin and amanitin toxin derivatives to pHLIP was shown to not change the pK of insertion (6.14 and 5.9, respectively).

Inhibition of Cell Proliferation. The ability of pHLIP to move MMAE across the cell membrane and to inhibit cancer cell proliferation in concentration and pH-dependent manners was evaluated. Cell treatments for both pHLIP(WT)-MMAE and pHLIP(D25E)-MMAE constructs were carried out with conjugate concentrations ranging from 1.25 to 10 μM at either pH 7.4 or 5.0. After a 2-hour incubation at 37° C., the treatment media was replaced by fresh media, and cells were grown for an additional 72 hours at normal pH before assessing cell viability using the MTT assay. This treatment protocol was chosen to prevent cell death that may result from prolonged exposure to low pH. This treatment protocol is quite stringent when compared to the typical multi-days exposure treatments used with free drugs (FIG. 6A and FIG. 6B).

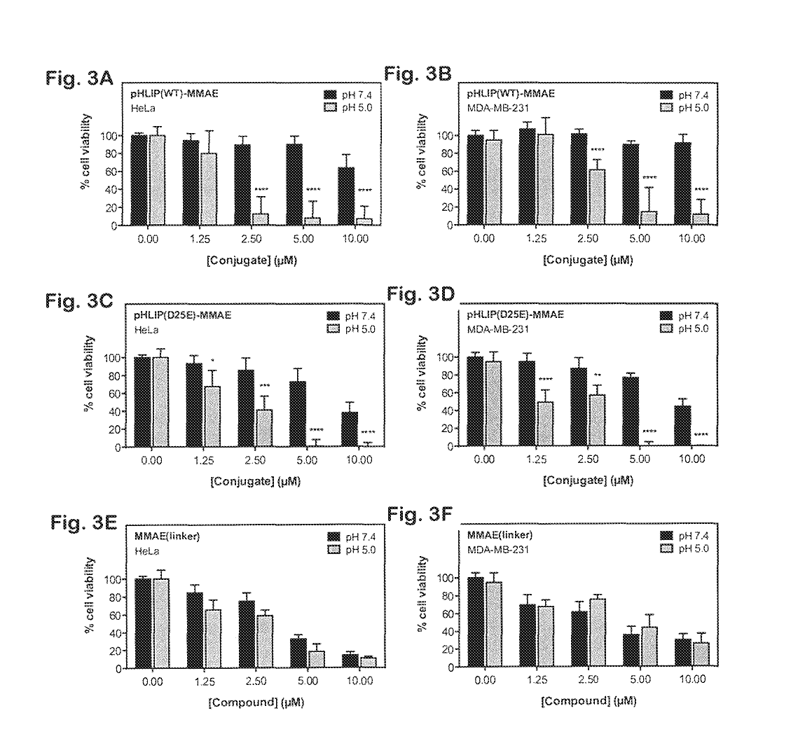

When HeLa cells were treated with pHLIP(WT)-MMAE at low pH, cell proliferation was severely disrupted. Up to 93% growth inhibition was observed (FIG. 3A). The anti-proliferative effect is concentration-dependent with cell viability ranging from 80% to 7%, with increasing treatment concentration ranging from 1.25 to 10 μM, respectively (FIG. 3A, grey bars). Inhibition of cell growth was also pH-selective: Treatment with pHLIP(WT)-MMAE at pH 7.4 under the same conditions had no or a moderate effect on cell proliferation (FIG. 3A; black bars). This lack of anti-proliferative effect at normal pH is consistent with the notion that delivery of MMAE is mediated by the pH-dependent insertion of pHLIP across the plasma membrane. The low pH treatment in itself did not have any detrimental effect on the proliferation of HeLa cells, as shown by treatment without pHLIP(WT)-MMAE (0 μM treatment).

pHLIP(WT)-MMAE was also tested with human MDA-MB-231 breast cancer cells (FIG. 3B). A similar concentration and pH-dependent anti-proliferative effect was observed: Up to 88% cell growth inhibition was observed with low pH treatment, while no significant inhibition of proliferation was observed for cells treated at normal pH. However, MDA-MB-231 seemed less sensitive to pHLIP(WT)-MMAE treatment, as shown by a higher cell viability with the 2.5 μM treatment. Without intending to be limited to any particular theory or mechanism of action, it is believed that this effect may be due to the apparent higher resistance of MDA-MB-231 cells to MMAE when compared to HeLa cells (FIGS. 6A and 6B).

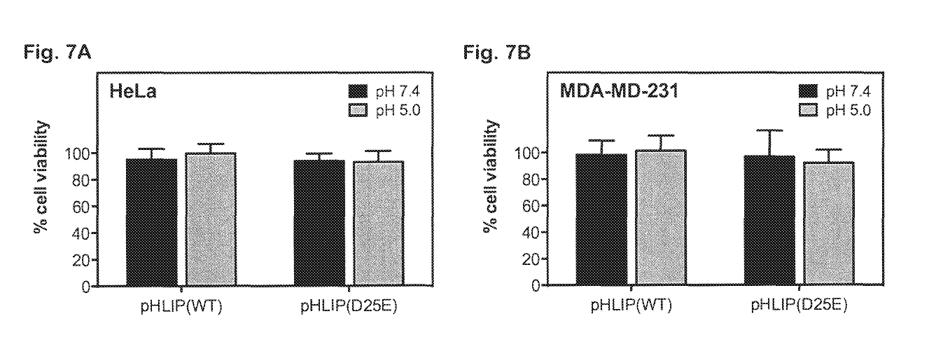

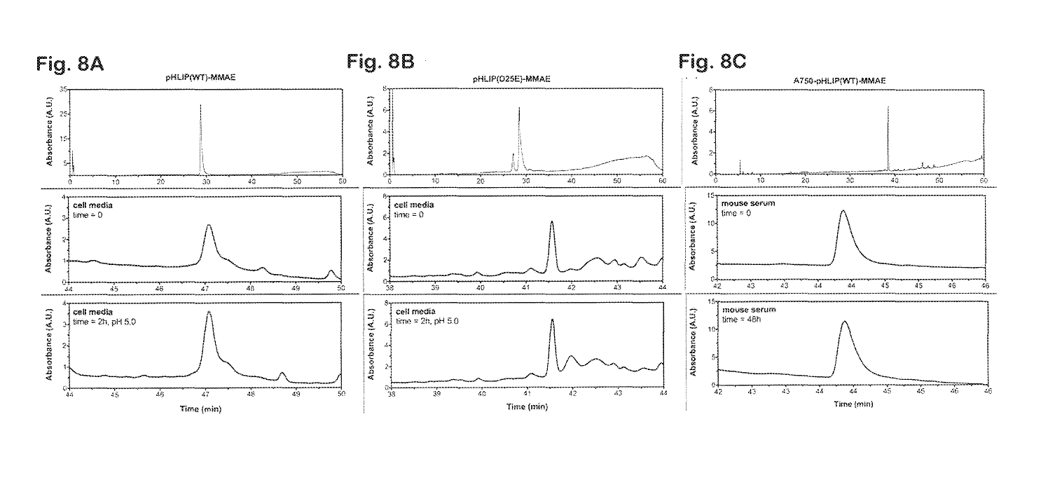

When HeLa and MDA-MB-231 cells were treated with pHLIP(D25E)-MMAE, cell proliferation was also disrupted in a concentration- and pH-dependent fashion (FIG. 3C and FIG. 3D). pHLIP(D25E)-MMAE appeared to be more effective at inhibiting cell growth than pHLIP(WT)-MMAE. It was especially evident when comparing treatments at 1.25 μM (FIG. 3C and FIG. 3D), with which greater cell killing was attained with pHLIP(D25E)-MMAE than with pHLIP(WT)-MMAE (MDA-MD-231 cells: 49% versus 100%, respectively). However, while 5 and 10 μM treatment exhibited nearly 100% growth inhibition at low pH (FIG. 3C and FIG. 3D; grey bars), non-negligible cytotoxicity was observed at pH 7.4, with about 75% and 40% cell viability, respectively (FIG. 3C and FIG. 3D; black bars). It is believed that this relatively high cytotoxicity at higher concentrations is not due to any intrinsic peptide cytotoxicity or to pHLIP insertion itself, as neither pHLIP(WT) or pHLIP(D25E) peptides have any harmful effects on the cell viability of either cell line at 10 μM (FIG. 7A and FIG. 7B). Cell killing did not seem to be due to instability of the linker (or any other chemical instability) in cell media either, as no degradation products are observed by HPLC or mass spectroscopy after low pH treatment with pHLIP(WT)-MMAE or pHLIP(D25E)-MMAE (FIG. 8).

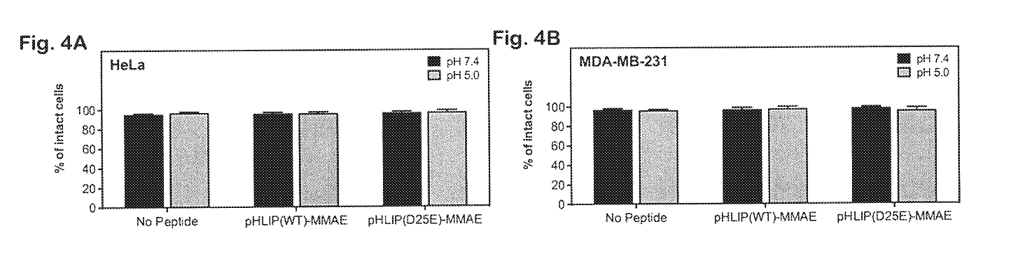

It was also tested whether either pHLIP-drug constructs may cause cell death through disruption of the plasma membrane: Cells were counted after a 2-hour treatment based on the uptake of trypan blue, which can only be taken up by cells if their plasma membrane is disrupted. FIG. 4A and FIG. 4B show that neither conjugates, nor low pH treatment, caused disruption of the membranes of either cell line. While pHLIP-mediated translocation of cargo molecules is thought to not be mediated by endocytosis, the possibility that the observed cytotoxicity of pHLIP(WT)-MMAE and pHLIP(D25E)-MMAE at higher concentrations might be associated with partial endocytotic uptake by the cells promoted by the interaction of pHLIP with the plasma membrane at low pH cannot be excluded.

Nevertheless, these results indicate that pHLIP-mediated translocation of MMAE can inhibit the proliferation of cancer cells in a pH-selective fashion, and offers clear advantages over treatment with free MMAE: (1) pHLIP prevents the high toxicity of MMAE at physiological pH. For instance, in FIG. 3A, 5 μM of pHLIP(WT)-MMAE shows only marginal cytotoxicity against HeLa whereas MMAE alone exhibits about 70% cytotoxicity (FIG. 3E). Similar results were observed with pHLIP(D25E)-MMAE and MDA-MB-231 cells. (2) Conjugation to pHLIP enhances MMAE anti-proliferative effects at low concentrations when treated at low pH. It was especially evident when comparing cell viability at 2.5 μL. While free MMAE has a marginal cytotoxicity effect at these concentrations (20-30%; FIGS. 3E and 3F), pHLIP-MMAE conjugates are systematically more toxic at low pH (up to 87.5% cytotoxicity; FIGS. 3A-FIG. 3D).

The results suggest that, on one hand, MMAE is effectively sequestered outside the cells by pHLIP at normal pH, preventing its passive diffusion into cells and its cytotoxicity. On the other hand, pHLIP improves MMAE potency by actively translocating it into the cytoplasm at lower pH in a mechanism more favorable (and/or faster) than passive diffusion, likely resulting in a higher effective MMAE concentration inside cells. All together, these effects participate in reducing the effective dose of MMAE necessary to observe cell death, which may alleviate off-target effect.

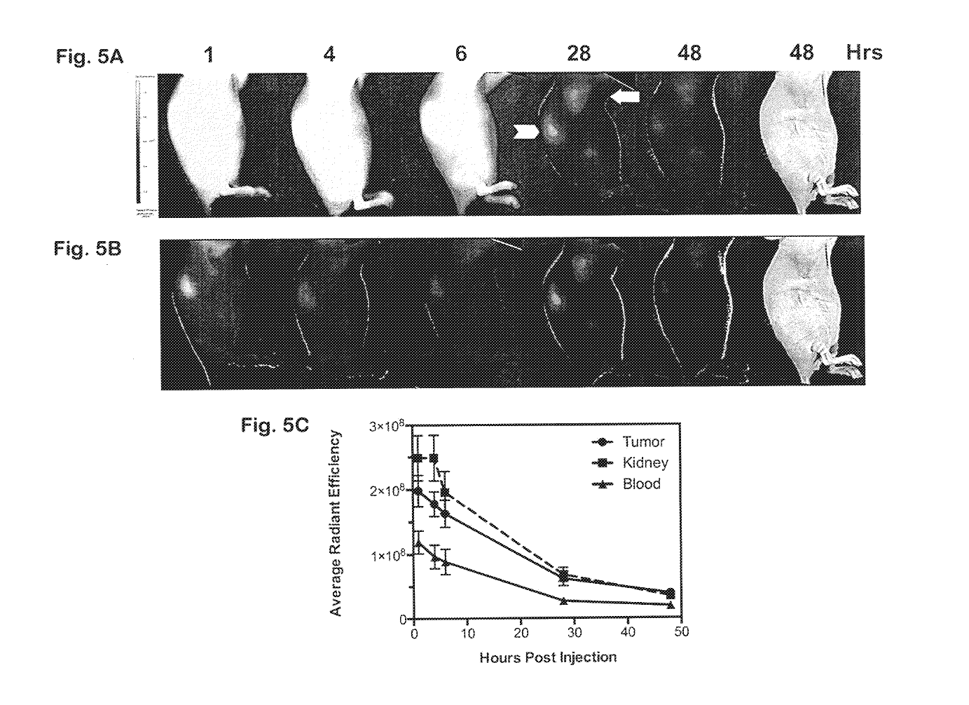

In vivo targeting. The pH-dependent anti-proliferative selectivity observed with pHLIP-MMAE and it low cytotoxicity at higher concentration in comparison with pHLIP(D25E)-MMAE prompted testing of the hypothesis that pHLIP can selectively deliver MMAE to tumors. As seen in FIG. 5A-FIG. 5C, intravenously administered Alexa750-pHLIP(WT)-MMAE was selectively retained in MDA-MB-231 breast cancer xenograft tumors over a 48 hour imaging time course. As tested, tumors were clearly visualized by 4-6 hours post-injection with maximum tumor: background ratios of 2.3 being observed at the 28 hour post-injection time point (FIG. 5C).

Consistent with a renal clearance mechanism, Alexa750-pHLIP(WT)-MMAE rapidly accumulates in kidney with concomitant clearance from the periphery. Background levels dropped 4.5-fold by 28 hours and 6-fold by 48 hours (FIG. 5C). The conjugation of MMAE to pHLIP did not discernibly alter the tumor targeting properties of pHLIP as compared to that seen with other Alexa-pHLIP constructs. The imaging studies were carried out with sub-therapeutic doses of Alexa750-pHLIP(WT)-MMAE to assess its tumor targeting capability and set the stage for in-depth studies aimed at determining the therapeutic efficacy and toxicity profiles associated with this novel drug conjugate.

The rapid and selective uptake of Alexa750-pHLIP(WT)-MMAE into tumors is analogous to that observed with rapidly cleared antibody-based agents. These data, coupled with the demonstration that pHLIP-MMAE is stable in serum over a relevant time frame for its systemic clearance rate suggests that appropriate dosing of pHLIP(WT)-MMAE will promote tumor control and limit the toxicities associated with free MMAE. As detailed above, the mechanism of action that drives tumor targeting of pHLIP-based constructs is independent of target antigen expression.

This Example shows that pHLIP provides localized delivery of MMAE, a clinically relevant cytotoxic agent. In combination, pHLIP-MMAE drug conjugates synergistically provide greater selectivity and inhibit cancer cell proliferation in a concentration- and pH-dependent manner in vitro. Moreover, the tumor homing abilities of pHLIP-MMAE conjugates were presented in MDA-MB-231 triple negative breast cancer xenograft tumors in vivo.

### Example 3

### Cytotoxicity of pHLIP-MMAF

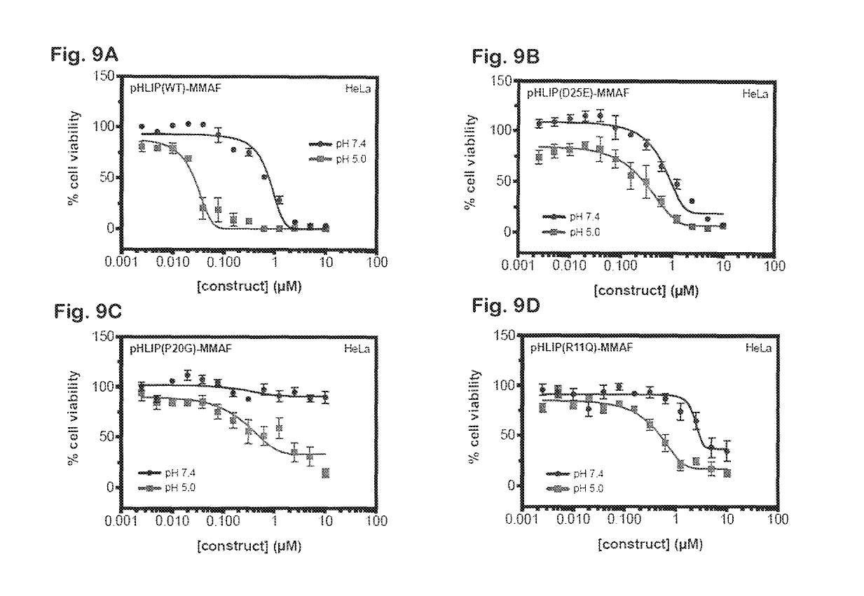

Preliminary cytotoxicity assays with cervical cancer HeLa cells using 4 variants of pHLIP-MMAF showed a clear pH-selective and concentration-dependent killing (FIG. 9A-FIG. 9D). HeLa cells were treated with pHLIP(WT)-MMAF (FIG. 9A), pHLIP(D25E)-MMAF (FIG. 9B), pHLIP(P20G)-MMAF (FIG. 9C), and pHLIP(R11Q)-MMAF (FIG. 9D). Circles represent cells treated at pH 7.4 and squares represent cells treated at pH 5.0. Results are shown as the mean±SEM (n=6-9).

However, pHLIP(WT)-MMAF showed the most efficacy with an estimated IC50 of approximately 0.02 microM. IT represents a significant improvement (ca. 100-fold) over treatments with pHLIP(WT)-MMAE, confirming MMAF as a cytotoxic choice over MMAE.

### Example 4

### Evaluation of pHLIP(WT)-MMAF for Therapeutic Efficacy in Mouse Models

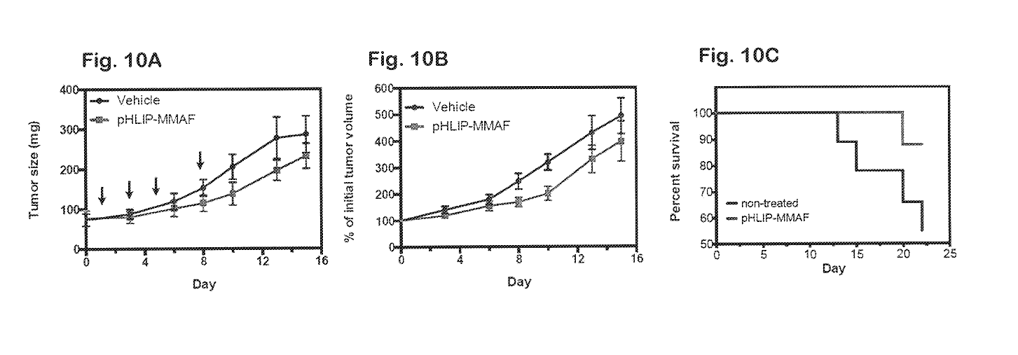

Intravenous injection of pHLIP-MMAF slowed growth of cervical cancer HeLa tumors when depicted both as absolute tumor weight and as a fraction of initial tumor size (FIG. 10A and FIG. 10B; see legend for FIG. 10 for experimental conditions). A tumor volume of 500 mm³ was prospectively set as the stopping point for the study. As depicted in FIG. 10C, treatment of mice with pHLIP-MMAF increased rates of survival as compared to the vehicle-treated cohort. At the end of the three week study, 4 animals in the control cohort had tumors that exceeded the 500 mm³ stopping point, and were removed from the study. This is in contrast to pHLIP-MMAF, where only a single animal had a tumor size that required its removal from the study.

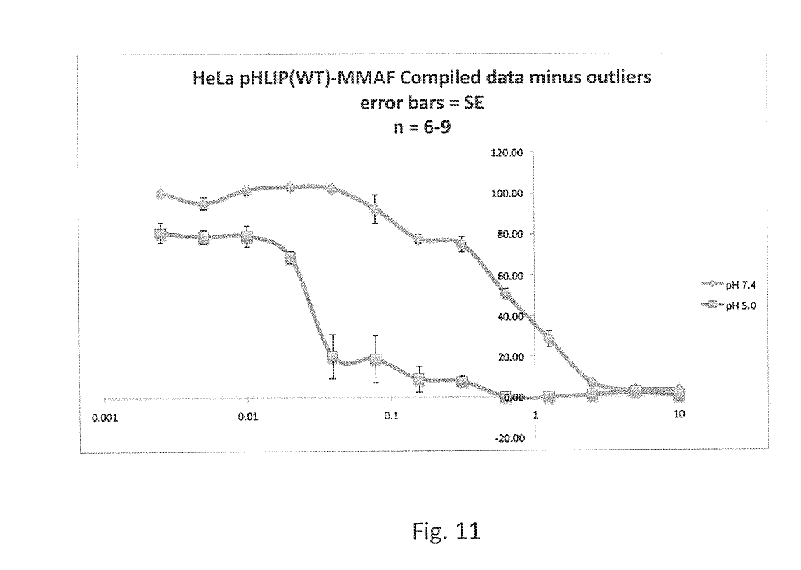

HeLa (ATCC#: CCL-2) were injected subcutaneously (s.c.) into the inguinal space of female Ncr nu/nu mice. Tumors were allowed to develop until they were approximately 75 mm³ in size. On Day 0 animals were randomized into cohorts with statistically similar tumor sizes; pHLIP-MMAF (76.8+/−19.3 mm³, n=8) and vehicle control (73+/−15.6 mm³, n=9) cohorts. On days 1, 3, 5, and 8 animals were injected intravenously with 200 microliters of either pHLIP-MMAF (20 microM in PBS+1% DMSO) or vehicle control (PBS+1% DMSO). Tumors were measured with calipers on routine basis for approximately 3 weeks and tumor volumes calculated with the formula Length×Width×(Height×0.5). Injection of pHLIP-MMAF slowed growth of HeLa tumors when depicted both as absolute tumor weight (FIG. 10A) and as a fraction of initial tumor size (FIG. 10B). A tumor volume of 500 mm³ was prospectively set as the stopping point for this study. As depicted in FIG. 11, treatment of mice with pHLIP-MMAF increased rates of overall survival as compared to the vehicle-treated cohort. At the end of the three-week study, 4 animals in the control cohort had tumors that exceeded the 500 mm³ stopping point and were removed from study. This is in contrast to pHLIP-MMAF were only a single animal had a tumor size that required its removal from study.

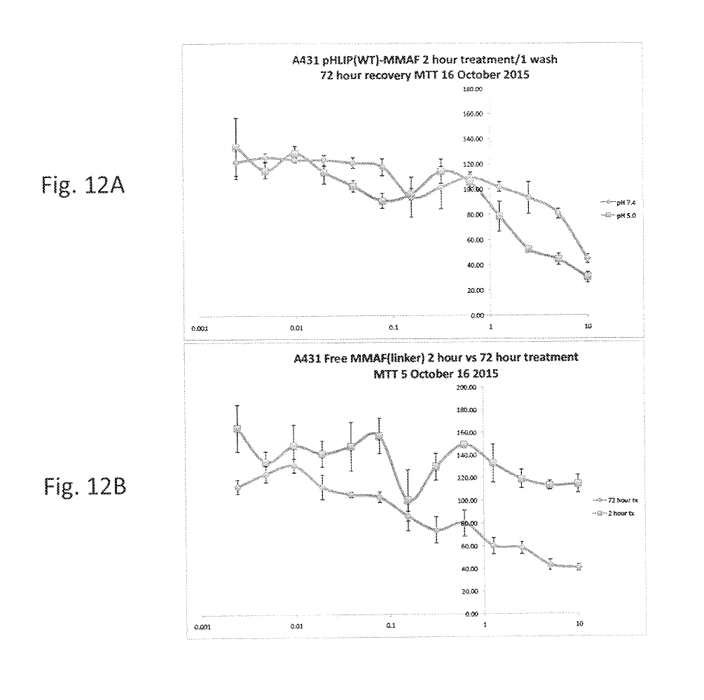

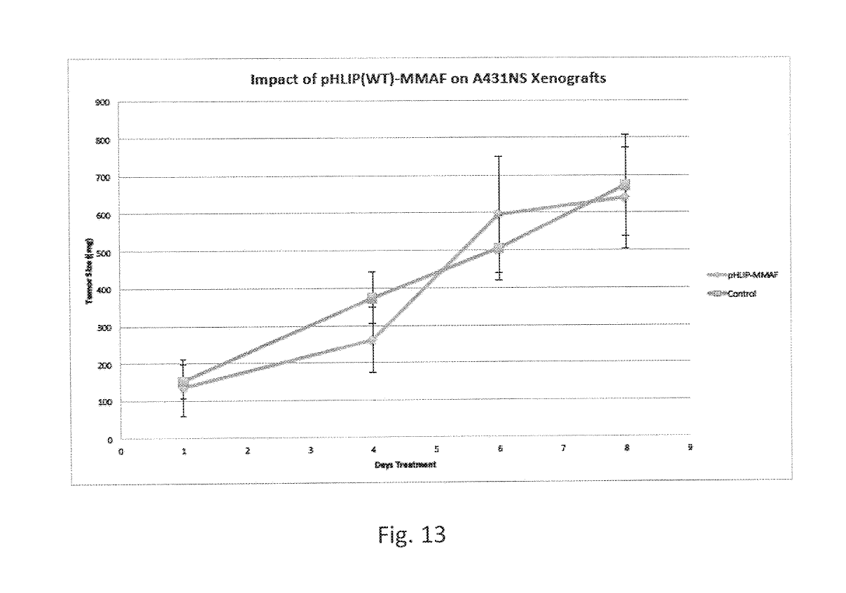

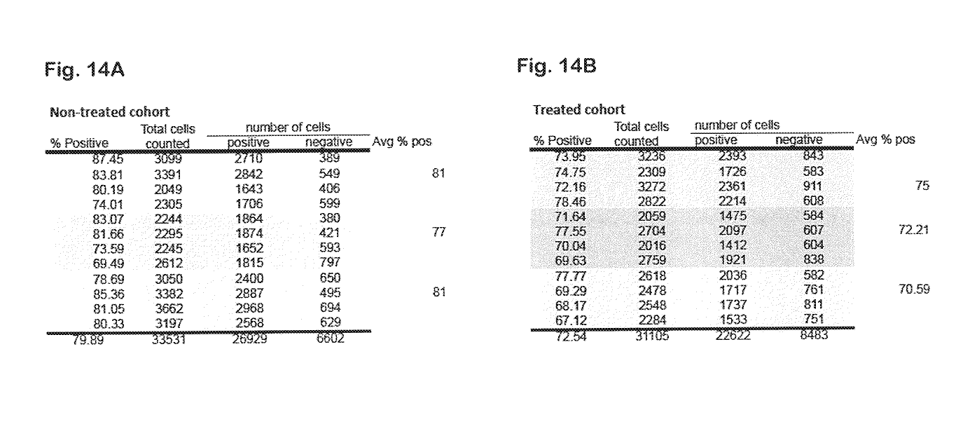

Analogous results were obtained when Ncr nu/nu mice bearing A431 (ATCC#: CRL-1555) tumors were treated i.p. with pHLIP-MMAF or vehicle control (FIGS. 12A and 12B, and FIG. 13). Immunohistochemistry analysis demonstrated that pHLIP-MMAF inhibited cell proliferation as compared to vehicle control when quantified by Ki-67. Wilcoxon ranked-sum analysis demonstrated that the difference was statistically significant (P<0.05) (FIG. 14A and FIG. 14B).

### Example 5

### Toxicity Studies

Four one milligram/kilogram (1 mg/kg) injections of pHLIP(WT)-MMAF are given to subjects on days 1, 3, 5 and 7. Overall health, liver and kidney function, and electrolytes are monitored using a complete blood count (CBC) and comprehensive metabolic panel, for a period of 2 weeks after the final injection. Clinically significant differences in overall health, liver and kidney function, and electrolytes were not observed between pre- and post-injection with placebo or pHLIP(WT)-MMAF. These results suggest that therapeutically effective amounts of the constructs of the present invention could be tolerated at doses above 1 mg/kg.

The invention is not limited to the embodiments described and exemplified above, but is capable of variation and modification within the scope of the appended claims.

Sequence Listing

SEQ ID NO: 1 (pHLIP WT)
AAEQNPIYWARYADWLFTTPLLLLDLALLVDADEGTCG
SEQ ID NO: 2 (pHLIP D25E)
AAEQNPIYWARYADWLFTTPLLLLELALLVDADEGTCG
SEQ ID NO: 3 (pHLIP P20G)
AAEQNPIYWARYADWLFTTGLLLLDLALLVDADEGTCG
SEQ ID NO: 4 (pHLIP R11Q)
AAEQNPIYWAQYADWLFTTPLLLLDLALLVDADEGTCG
SEQ ID NO: 5 (pHLIP R11Q + D14UP)
AAEQNPIYWAQYADAWLFTTPLLLLDLALLVDADEGTCG
SEQ ID NO: 6 (D14Gla + D25Aad)*
AAEQNPIYWARYAWLFTTPLLLLXLALLVDADEGTCG
*X at position 14 replaces D with Gla = gamma-
carboxyglutamic acid
*X at position 25 replaces D with Aad = alpha-
aminoadipic acid
SEQ ID NO: 7 (pHLIP WT + C-terminal Cys)
AAEQNPIYWARYADWLFTTPLLLLDLALLVDADEGTCGC
SEQ ID NO: 8 (pHLIP consensus)
AAEQNPIYWAXYAWLFTTXLLLLXLALLVDADEGTCG
X at position 11 is R or Q
X at position 14 is D or Gla (gamma-carboxy-
glutamic acid)
X at position 20 is P or G
X at position 25 is D, E, Aad (alpha-aminoadipic
acid)
SEQ ID NO: 9 (pHLIP consensus + C-terminal Cys)
AAEQNPIYWAXYAWLFTTXLLLLXLALLVDADEGTCGC
X at position 11 is R or Q
X at position 14 is D or Gla (gamma-
carboxyglutamic acid)
X at position 20 is P or G
X at position 25 is D, E, or Aad (alpha-
aminoadipic acid)

1-70. (canceled)

71. A construct, comprising a pH Low Insertion Peptide (pHLIP) comprising the amino acid sequence of SEQ ID NO: 8 or SEQ ID NO: 5, conjugated to a compound of Formula (I) 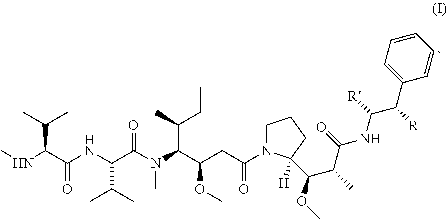
wherein R is H or OH, and R′ is C₃H₃NS, CH₃, a carboxylic acid, or an alkyl ester.

72. The construct according to claim 71, wherein the pHLIP comprises the amino acid sequence of SEQ ID NO: 1.

73. The construct according to claim 71, wherein the pHLIP comprises the amino acid sequence of SEQ ID NO: 2.

74. The construct according to claim 71, wherein the pHLIP comprises the amino acid sequence of SEQ ID NO: 3.

75. The construct according to claim 71, wherein the pHLIP comprises the amino acid sequence of SEQ ID NO: 4.

76. The construct according to claim 71, wherein the pHLIP comprises the amino acid sequence of SEQ ID NO: 6.

77. The construct according to claim 71, wherein the pHLIP comprises the amino acid sequence of SEQ ID NO: 7.

78. The construct according to claim 71, wherein the pHLIP comprises the amino acid sequence of SEQ ID NO: 9.

79. The construct according to claim 71, wherein the R is OH and R′ is CH₃.

80. The construct according to claim 71, wherein the R is H and R′ is a carboxylic acid.

81. The construct according to claim 80, wherein the carboxylic acid is acetic acid.

82. The construct according to claim 71, wherein the R is H and R′ is an alkyl ester.

83. The construct according to claim 82, wherein the alkyl ester is methyl ester.

84. The construct according to claim 71, wherein the pHLIP and the compound are conjugated via a disulfide bond.

85. A composition, comprising the construct according to claim 1; and a pharmaceutically acceptable carrier.

86. A method for inhibiting proliferation of a tumor cell, comprising contacting the cell with an effective amount of a construct according to claim 1.

87. The method according to claim 86, wherein the tumor cell is a breast tumor cell, a prostate tumor cell, a pancreatic tumor cell, a cervical tumor cell, a uterine tumor cell, a lung tumor cell, a skin cancer cell, a kidney cancer cell, or a colon tumor cell.

88. The method according to claim 86, further comprising administering a chemotherapeutic agent to the patient or irradiating the tumor in the patient.

89. The method according to claim 86, wherein the tumor cell is in an environment having a pH of less than about 7.

90. A kit, comprising a composition comprising a pharmaceutically acceptable carrier and the construct according to claim 71, and instructions for using the composition in a method for treating breast cancer.


SEQUENCE LISTING (9 sequences)

SEQ ID NO: 1 - protein, 38 aa, Halobacterium salinarum
AAEQNPIYWARYADWLFTTPLLLLDLALLVDADEGTCG

SEQ ID NO: 2 - protein, 38 aa, artificial
  artificial peptide pHLIP D25E derived from Halobacterium salinarum pHLIP where the Asp (D) 25 is replaced with a Glutamic acid (E)
AAEQNPIYWARYADWLFTTPLLLLELALLVDADEGTCG

SEQ ID NO: 3 - protein, 38 aa, artificial sequence
  artificial peptide pHLIP P20G derived from Halobacterium salinarum pHLIP where the proline (P) at 20 is replaced with a Glycine (G)
AAEQNPIYWARYADWLFTTGLLLLDLALLVDADEGTCG

SEQ ID NO: 4 - protein, 38 aa, artificial sequence
  artificial peptide pHLIP R11Q derived from Halobacterium salinarum pHLIP where the arginine (R) at 11 is replaced with a Glutamine (Q)
AAEQNPIYWAQYADWLFTTPLLLLDLALLVDADEGTCG

SEQ ID NO: 5 - protein, 39 aa, artificial sequence
  artificial peptide pHLIP R11Q + D14UP derived from Halobacterium salinarum pHLIP where the Arginine (R) at 11 is replaced with a Glutamine (Q) and an additional Alanine (A) is inserted following the Aspartic Acid (D) at 14
AAEQNPIYWAQYADAWLFTTPLLLLDLALLVDADEGTCG

SEQ ID NO: 6 - protein, 38 aa, artificial sequence
  artificial peptide D14Gla + D25Aad derived from Halobacterium salinarum pHLIP where the Aspartic Acid (D) at 14 is replaced with a gamma-carboxyglutamic acid (Gla) and the Aspartic Acid (D) at 25 is replaced with an alpha-aminoadipic acid (Aad)
  D14Gla (14)..(14): the X is a gamma-carboxyglutamic acid in lieu of Aspartic acid (D)
  D25Aad (25)..(25): the X is an alpha-aminoadipic acid in lieu of aspartic acid
AAEQNPIYWARYAXWLFTTPLLLLXLALLVDADEGTCG

SEQ ID NO: 7 - protein, 39 aa, artificial sequence
  artificial peptide derived from Halobacterium salinarum pHLIP including a C-terminal cysteine (C)
AAEQNPIYWARYADWLFTTPLLLLDLALLVDADEGTCGC

SEQ ID NO: 8 - protein, 38 aa, Halobacterium salinarum
  x11 (11)..(11): X at position 11 is Arginine (R) or Glutamine (Q)
  X14 (14)..(14): X at position 14 is aspartic acid (D) or gamma-carboxyglutamic acid (Gla)
  X20 (20)..(20): X at position 20 is Proline (P) or Glycine (G)
  X25 (25)..(25): X at position 25 is Aspartic Acid (D), Glutamic acid (E), or alpha-aminoadipic acid (Aad)
AAEQNPIYWAXYAXWLFTTXLLLLXLALLVDADEGTCG

SEQ ID NO: 9 - protein, 39 aa, artificial sequence
  artificial peptide derived from Halobacterium salinarum pHLIP consensus including a C-terminal cysteine (C)
  x11 (11)..(11): X at position 11 is Arginine (R) or Glutamine (Q)
  x14 (14)..(14): X at position 14 is aspartic acid (D) or gamma-carboxyglutamic acid (Gla)
  x20 (20)..(20): X at position 20 is Proline (P) or Glycine (G)
  x25 (25)..(25): X at position 25 is Aspartic Acid (D), Glutamic acid (E), or alpha-aminoadipic acid (Aad)
AAEQNPIYWAXYAXWLFTTXLLLLXLALLVDADEGTCGC